(19) United States
(12) Patent Application Publication
(10) Pub. No.: US 2009/0030066 A1
(43) Pub. Date: Jan. 29, 2009

(54) SMALL MOLECULES FOR THE PROTECTION OF PANCREATIC CELLS

(75) Inventor: Zoltan Kiss, Austin, MN (US)

Correspondence Address: FAEGRE & BENSON LLP;PATENT DOCKETING, 2200 WELLS FARGO CENTER, 90 SOUTH SEVENTH STREET, MINNEAPOLIS, MN, 55402-3901, US

(73) Assignee: Zoltan Laboratories LLC, Austin, MN (US)

(21) Appl. No.: 12/178,282

(22) Filed: Jul. 23, 2008

Related U.S. Application Data
(60) Provisional application No. 60/951,341, filed on Jul. 23, 2007.

(51) Int. Cl. A61K 31/382; A61K 31/33; A61P 37/06

(52) U.S. Cl. 514/437; 514/183

(57) ABSTRACT

Embodiments of the present invention include the in vivo and in vitro use of a family of anticancer heterocyclic compounds containing a quaternary ammonium group as exemplified by the thioxanthone and thioxanthene compounds [3-(3,4-dimethyl-9-oxo-9H-thioxanthen-2-yloxy)-2-hydroxypropyl]trimethylammonium chloride, or CCompound1, N,N,-diethyl-N-methyl-2-[9-oxo-9H-thioxanthen-2-yl)methoxy]ethanaminium iodide, or CCompound3, and N,N,N-trimethyl-3-(9H-thioxanthen-9-ylidene)-propane-1-aminium iodide, or CCompound19 to maintain and increase viability of normal endocrine and exocrine pancreatic cells under pathological conditions, such as type 1 and type 2 diabetes, pancreatitis, pancreatic cancer, or during and after islet transplant, or in preparation for transplant of isolated islet cells via (i) direct contact with these cells, and/or via (ii) enhancing survival and proliferation of endogenous or transplanted adult stem cells, and/or via (iii) reducing viability of pancreatic cancer cells.

### RELATED APPLICATIONS

This application claims the benefit under 35 U.S.C. §119 of U.S. Provisional Application No. 60/951,341, filed Jul. 23, 2007, which is herein incorporated by reference in its entirety.

### TECHNICAL FIELD

The invention provides a family of anticancer heterocyclic compounds containing a quaternary ammonium group as exemplified by the thioxanthone and thioxanthene compounds [3-(3,4-dimethyl-9-oxo-9H-thioxanthen-2-yloxy)-2-hydroxypropyl]trimethylammonium chloride, or CCompound1, N,N,-diethyl-N-methyl-2-[9-oxo-9H-thioxanthen-2-yl)methoxy]ethanaminium iodide, or CCompound3, and N,N,N-trimethyl-3-(9H-thioxanthen-9-ylidene)-propane-1-aminium iodide, or CCompound19 to maintain or increase viability of endocrine pancreatic cells, such as β-cells, and exocrine pancreatic cells in vitro or under pathological conditions in vivo.

### BACKGROUND

Extensive destruction of the endocrine insulin producing islet β-cells in the pancreas is the hallmark of type 1 or insulin-dependent diabetes. However, major loss of islet cells also frequently occurs in aging and diseased subjects such as those suffering from chronic inflammation of pancreas (pancreatitis), pancreatic cancer, or type 2 diabetic subjects. Islet cell loss in type 1 diabetic and type 2 diabetic patients on average is about 98% and 60%, respectively.

One consequence of reduced secretion of insulin into the blood is elevated blood glucose level. In turn, higher than normal levels of glucose in the blood accelerate the destruction of the remaining islet cells. The destructive effects of high glucose are mediated by reactive oxygen species (ROS) often involving pro-apoptotic cytokines [Wu, L., Nicholson, W., Knobel, S. M., Stefffier, R. J., May, J. M., Piston, D. W. and Powers, A. C. (2004) Oxidative stress is a mediator of glucose toxicity in insulin-secreting islet cell lines. J. Biol. Chem. 279, 12126-12134; Tabatabaie, T., Vasquez-Weldon, A., Moore, D. R. and Kotake, Y. (2003) Free radicals and the pathogenesis of type 1 diabetes: β-cell cytokine-mediated free radical generation via cyclooxygenase-2. Diabetes 52, 1994-1999; Roberston, R. P., Harmon, J., Tran, P. O., Tanaka, Y. and Takahashi, H. Glucose toxicity in β-cells: Type 2 diabetes, good radicals gone bad, and the glutathione connection (2003) Diabetes 52, 581-587]. The result is further reduction of insulin and therefore still higher levels of glucose in the patient's blood. Dangerously high levels of glucose in the blood, or hyperglycemia, may lead to diabetes [Mandrup-Poulsen, T. (2001) β-cell apoptosis. Diabetes 50, (Suppl. 1): S58-S63].

Higher than normal levels of saturated free fatty acids in the circulation may also lead to islet cell dysfunction and destruction via apoptosis mediated by ROS, excess nitric oxide (NO), and ceramide that affect downstream cellular events involved in the regulation of cell viability [Lupi, R., Dotta, F., et al. (2002) Prolonged exposure to free fatty acids has cytostatic and pro-apoptotic effects on human pancreatic islets. Diabetes 51, 1437-1442; Rachek, L. I., Thornley, N. P., Grishko, V. I., LeDoux, S. P. and Wilson, G. L. (2006) Protection of INS-1 cells from free fatty acid-induced apoptosis by targeting hOGG1 to mitochondria. Diabetes 55, 1022-1028].

Thus, elevated levels of glucose and free saturated fatty acids in the blood both play significant roles in ROS and NO-mediated β-cell dysfunction and development of diabetes leading to complications in target organs including cardiomyopathy, nephropathy, retinopathy, and peripheral neuropathy among others [Evans, J. L., Goldfine, I. D., Maddux, B. A. and Grodsky, G. M. (2003) Are oxidative stress-activated signaling pathways mediators of insulin resistance and β-cell dysfunction? Diabetes 52, 1-8; Green, K., Brand, M. D. and Murphy, M. P. (2004) Prevention of mitochondrial oxidative damage as a therapeutic strategy in diabetes. Diabetes 53 (Suppl. 1): S110-S118].

Pancreatitis is characterized by a certain level of inflammation-induced destruction of parenchymal tissue leading to the loss or reduction of exocrine and endocrine functions. Inflammation is triggered by increased levels of inflammatory cytokines, such as tumor necrosis factor-α, interferon-γ, interleukin-1, and interleukin-6, released from recruited inflammatory cells. Since these cytokines are known to increase the formation of NO and ROS, it is generally assumed that free radicals play an important role in the development of pancreatitis [Krajewski, E., Krajewski, J., Spodnik, J. H., Figarski, A. and Kunasik-Juraniec, J. (2005) Changes in the morphology of the acinar cells of the rat pancreas in the oedematous and nectoric types of experimental acute pancreatitis. Folia Morphol. 64, 292-303]. Pancreatitis also affects β-cell function, in addition to other types of pancreatic cells. In pancreatic cancer patients both islet β-cell dysfunction and insulin resistance can occur. Furthermore, among other possible causes distal pancreatitis has been implicated in β-cell dysfunction [Noy, A. and Bilezikian, J. P. (1994) Clinical Review 63; Diabetes and pancreatic cancer: Clues to the early diagnosis of pancreatic malignancy. J. Clin. Endocrinol. Met. 79, 1223-1231; and references therein].

Diabetic patients are presently treated with insulin and/or other anti-diabetic agents. In severe cases of hyperglycemia, when a patient's islet cells are destroyed so extensively that survival requires frequent administration of insulin, islet cells may be transplanted into the patient. However, short supply of islet cell donors and inactivation of islet functions during the isolation process and following transplantation seriously limits this form of therapy. Treatment with an antioxidant may improve the yield and the function of transplanted human islets [Bottino, R., Balamurugan, A. N., Tse, H., Thirunavukkarasu, C., Ge, X., Profozich, J., Milton, M., Ziegenfuss, A., Trucco, M. and Piganelli, J. D. (2004) Response of human islets to isolation stress and the effect of antioxidant treatment. Diabetes 53, 2559-2568]. However, there is currently no known clinical method utilizing an antioxidant to protect islets in vitro or in vivo.

Several other agents have been described to promote islet cell survival including the incretin glucagon-like peptide analogues [Holst, J. J. and Orskov, C. (2004) The incretin approach for diabetes treatment. Diabetes 53, (Suppl. 3): S197-S204], inhibitors of glycogen synthase kinase3 [Mussmann, R., Geese, M., Harder, F., Kegel, S., Andag, U., Lomow, A., Burk, U., Onichthouk, D., Dohrmann, C. and Austen, M. (2007) Inhibition of GSK3 promotes replication and survival of pancreatic beta cells. J. Biol. Chem. 282, 12030-12037], α1-antitrypsin [Lewis, E. C., Shapiro, L., Bowers, O. J. and Dinarello, C. A. (2005) α1-antitrypsin monotherapy prolongs islet allograft survival in mice. Proc. Natl. Acad. Sci. USA, 23, 12153-12158], a caspase inhibitor [Emamaullee, J. A., Stanton, L., Schur, C. and Shapiro, A. M. J. (2007) Caspase inhibitor therapy enhances marginal mass islet graft survival and preserves long-term function in islet transplantation. Diabetes 56, 1289-1298], 3,5,3′-triiodothyronine [Falzacappa, C. V., Panacchia, L., Bucci, B., Stigliano, A., Cavallo, M. G., Brunelli, E., Toscano, V. and Misiti, S. (2006) 3,5,3′-triiodothyronine (T3) is a survival factor for pancreatic β-cells undergoing apoptosis. J. Cell. Pathol. 206, 309-321], epidermal growth factor in combination with gastrin [Suarez-Pinzon, W. L., Yan, Y., Power, R., Brand, S. J. and Rabinovitch, A. (2005) Combination therapy with epidermal growth factor and gastrin increases β-cell mass and reverses hyperglycemia in diabetic NOD mice. Diabetes, 54, 2596-2601], inhibitors of NFκB activation such as pioglitazone and sodium salicylate [Zeender, E., Maedler, K., Bosco, D., Bemey, T., Donath, M. Y. and Halban, P. A. (2004) Pioglitazone and sodium salicylate protect human β-cells against apoptosis and impaired function induced by glucose and interleukin-1β. J. Clin. Endocrinol. Metabol. 89, 5059-5066], and placental alkaline phosphatase. So far only pioglitazone and a related thiazolidinedione compound, rosiglitazone, have been shown in humans to improve β-cell function in humans [Gastaldelli, A., Ferrannini, E., Miyazaki, Y., Matsude, M., Mari, A. and DeFronzo, R. A. (2006) Thiazolidinediones improve β-cell function in type 2 diabetic patients. Am. J. Physiol. Endocrinol. Metab. 292, E871-E883]. However, a potential drawback of using thiazolidinedione compounds is that they significantly increase fat mass and BMI [Gastaldelli, A., Ferrannini, E., Miyazaki, Y., Matsude, M., Mari, A. and DeFronzo, R. A. (2006) Thiazolidinediones improve β-cell function in type 2 diabetic patients. Am. J. Physiol. Endocrinol. metab. 292, E871-E883]; in addition, most recent studies indicate that they may also increase the risk of heart attacks.

Adult stem cells, primarily mesenchymal stem cells (MSCs) derived from the bone marrow or cord blood, have recently been shown to possess the capacity to aid regeneration of damaged tissues such as skin, ischemic brain, muscle and myocardium as well as enhance engraftment of hematopoietic stem cells [Pittenger, M. F., Mackay, A. M., Beck, S. D., Jaiswal, R. K., Douglas, R., Mosca, J. D., Moorman, M. A., Simonetti, D. W., Craig, S, and Marschak, D. R. (1999) Multilineage potential of adult human mesenchymal stem cells. Science 284, 143-147; Baksh, D., Song, L. and Tuan, R. S. (2004) Adult mesenchymal stem cells: characterization, differentiation, and application in cell and gene therapy. J. Cell. Mol. Med. 8, 301-316; McFarlin, K., Gao, X., Liu, Y. B., Dulchavsky, D. S., Kwon, D., Arbab, A. S., Bansal, M., Li, Y., Chopp, M., Dulchavsky, S. A. and Gautam, S. C. (2006) Bone marrow-derived mesenchymal stemm cells accelerate wound healing in the rat. Wound Rep. Reg. 14, 471-478; Xiao, J., Nan, Z., Motooka, Y. and Low, W. C. (2005) Transplantation of a novel cell line population of umbilical cord blood cells ameliorates neurological deficits associated with ischemic brain injury. Stem Cell Develop. 14, 722-733; Dezawa, M., Ishikawa, H., Itokazu, Y., Yoshihara, T., Hoshino, M., Takeda, S. I., Ide, C. and Nabeshima, Y. I. (2005) Bone marrow stromal ells generate muscle cells and repair muscle degeneration. Science 309, 314-317; Nishiyama, N., Miyoshi, S., Hida, N., Uyama, T., Okamoto, K., Ikegami, Y., Miyado, K., Segawa, K., Terai, M., Sakamoto, M., Ogawa, S, and Umezawa, A. (2007) The significant cardiomyogenic potential of human umbilical cord blood-derived mesenchymal stem cells in vitro. Stem Cells 25, 2017-2024; Berry, M. F., Engler, A. J., Woo, Y. J., Pirolli, T. J., Bish, L. T., Jayasankar, V., Morine, K. J., Gardner, T. J., Discher, D. E. and Sweeney, H. L. (2006) Mesenchymal stem cell injection after myocardial infarction improves myocardial compliance. Am. J. Physiol. Heart Circ. Physiol. 290, H2196-H2203; Miyahara, Y., Nagaya, N., Kataoka, M., Yanagawa, B., Tanaka, K., Hao, H., Ishino, K., Ishida, H., Shimizu, T., Kangawa, K., Sano, S., Okano, T., Kitamura, S, and Mori, H. (2006) Monolayered mesenchymal stem cells repair scarred myocardium after myocardial infarction. Nature Medicine 12, 459-465; and Blanc, K. L., Samuelsson, H., Gustafsson, B., Remberger, M., Sundberg, B., Arvidson, J., Ljungman, P., Lonnies, H., Nava, S, and Ringden, O. (2007) Transplantation of mesenchymal stem cells to enhance engraftment of hematopoietic stem cells. Leukemia 21, 1733-1738].

A particularly useful property of MSCs is that they avoid allogeneic rejection [Barry, F. P., Murphy, J. M. English, K. and Mahon, B. P. (2005) Immunogenicity of adult mesenchymal stem cells: Lessons from the fetal allograft. Stem Cells Develop. 14, 252-265] which makes them ideal candidates for regenerative medicine for which examples were provided above. Because of the good toleration of MSCs by the host, these cells can also serve as vehicles for delivery of genes for gene therapy [Zhou, H., Ramiya, V. K. and Visner, G. A. (2006) Bone marrow stem cells as a vehicle for delivery of heme oxygenase-1 gene. Stem Cells Develop. 15, 79-86].

Of particular relevance to the present topic is the finding that MSCs and bone marrow cells cooperate to promote regeneration of islet cells in type 1 diabetic models [Urban, V. S., Kiss, J., Kovacs, J., Gocza, E., Vas, V., Monostori, E. and Uher, F. (2008) Mesenchymal stem cells cooperate with bone marrow cells in therapy of diabetes. Stem Cells 26, 244-253].

### SUMMARY OF THE INVENTION

Experimental animal models of diabetes and pancreatitis were developed by using streptozotocin and L-arginine, respectively. In both cases, islet cell death as well as the death of other types of pancreatic cells, just like in most pathological conditions, is known to occur by free radical-mediated mechanisms [Szkudelski, T. (2001) The mechanism of alloxan and streptozotocin action in β-cells of the rat pancreas. Physiol. Rev. 50, 536-546; Krajewski, E., Krajewski, J., Spodnik, J. H., Figarski, A. and Kunasik-Juraniec, J. (2005) Changes in the morphology of the acinar cells of the rat pancreas in the oedematous and nectoric types of experimental acute pancreatitis. Folia Morphol. 64, 292-303]. It is shown that CC compounds are suitable to reduce free radical-induced damage to the function of islet cells and other pancreatic cells. In addition, data are presented to indicate that in vitro, concentrations of CC compounds that correspond to physiologically effective amounts enhance survival and proliferation of MSCs while inhibiting the proliferation of pancreatic tumor cells. The results show that CC compounds are useful to directly protect islet β-cells and other types of pancreatic cells under such pathological conditions as type 1 and type 2 diabetes, pancreatitis, and pancreatic cancer, and they may promote regeneration of damaged β-cells and other damaged cells indirectly via enhancing the viability of MSCs.

The present invention relates to the use of heterocyclic compounds containing a quaternary ammonium group as exemplified by the thioxanthone and thioxanthene compounds [3-(3,4-dimethyl-9-oxo-9H-thioxanthen-2-yloxy)-2-hydroxypropyl]trimethylammonium chloride, or CCompound1, N,N,-diethyl-N-methyl-2-[9-oxo-9H-thioxanthen-2-yl)methoxy]ethanaminium iodide, or CCompound3, and N,N,N-trimethyl-3-(9H-thioxanthen-9-ylidene)-propane-1-aminium iodide, or CCompound19 to maintain or increase viability of insulin producing islet β-cells, other pancreatic cells, and adult stem cells in vitro or under pathological conditions in vivo.

For example, CCompound1 and CCompound19 protected islet β-cells in the streptozotocin (STZ)-induced type 1 diabetes model. In the L-arginine-induced necrotic pancreatitis mouse model, CCompound1 partially or fully protected both the endocrine and exocrine pancreatic cells. In the STZ-treated animals CCompound3 also clearly protected β-cell function. Protection of pancreatic cells includes, but is not limited to, protection against free radical-induced damage that accounts for the inhibitory effects of STZ, high glucose, and saturated fatty acids on the viability of islet cells.

In this application, the term “islet protection” means that the agent used reduces the death of β-cells in vivo and in vitro and thereby promotes their expansion under conditions that otherwise induce the death of islet cells. Protection of islet cell viability enhances the capacity of islet β-cells to increase insulin release in response to meal challenge. It is also assumed that the ability of agents such as CC compounds to reduce the enhancing effects of STZ or L-arginine on blood glucose level is related, at least in part, to the ability of such agent to protect the viability of β-cells. The term “protection of pancreatic cells” means that the agentreduces the death of all pancreatic cells under inflammatory conditions such as, for example, pancreatitis and, to some extent, pancreatic cancer. Such protection, which can result in less reduction or even the expansion of the number of pancreatic cells, is the combination of direct protecting effects of CC compounds on the pancreatic cells and protection via promoting the viability of stem cells such as MSCs. The extent of contribution of stem cells to islet regeneration will depend on specific conditions such as the extent and rate of islet cell death. However, it is likely that under any pathological condition both the direct effects and stem cell-mediated effects of a CC compound are involved in the protection of islet and other pancreatic cells.

As reviewed above, adult stem cells, including bone marrow derived MSCs, play key roles in tissue regeneration. In addition, reduction in the number of islet cells and other pancreatic cells that leads to the diabetic state is generally accompanied by the damage of other tissues as well. For example, the diabetic state is often the cause of associated diseases such as cardiovascular disease, cardiomyopathy, neuropathy, nephropathy, retinopathy, and impaired wound healing. Regeneration of the corresponding tissues involves adult stem cells and progenitor cells often migrated from the bone marrow. Thus, application of a CC compound is expected to improve stem cell based regeneration of various tissues damaged as a consequence of the diabetic state that, in turn, results from deterioration of islet cell viability. While the protective actions of CC compounds include various tissues affected by the diabetic state, for simplicity, in the rest of the text the focus will be on the effects of CC compounds on the viability of pancreatic cells, stem cells, and pancreatic cancer cells.

Further, CC compounds protect normal pancreatic cells while they inhibit the proliferation of pancreatic cancer cells. This can be exploited when the treated subject has pancreatic cancer associated with islet dysfunction.

CC compounds may be administered, alone or along with other protective agents, to a patient with type 1 diabetes, type 2 diabetes, pancreatitis, or pancreatic cancer with associated islet dysfunction to enhance survival of remaining β-cells and other pancreatic cells attacked by high glucose, saturated fatty acids, inflammatory conditions, and/or ROS/NO. CC compounds may also be used to treat patients who received transplanted islet cells with and without stem cell support to protect these cells in vitro as well as in vivo against ROS/NO-mediated attacks by the patient's immune system. Some results of in vivo protective effects of CC compounds are increased insulin secretion, better control of blood glucose level, and better protection of tissues affected by the diabetic state. Finally, CC compounds may also be used in vitro during or after preparation of islet cells and/or stem cells for transplantation to type 1 diabetic patients.

In some embodiments, the invention includes a method of enhancing the viability of islet and other pancreatic cells thereby promoting insulin secretion in a mammal by administering a CC compound alone or together with another promoter of islet cell viability to the mammal. In some such embodiments, the islet cells may be transplanted into the mammal with or without stem cell support and isolated islet cells and the stem cells may be treated with the CC compound during the isolation and transplantation processes and/or after the transplantation. In special cases only adult stem cells are transplanted to improve viability of endogenous islet cells and any other cell types that are affected by the diabetic state.

In other embodiments, the invention provides a treatment regimen for the treatment of a mammal with type 1 diabetes, type 2 diabetes, pancreatitis, or pancreatic cancer with decreasing islet cell viability comprising periodically administering a therapeutically effective amount of CC compound alone or together with another promoter of islet cell survival.

In yet further embodiments, the invention provides for the use of a CC compound alone or together with another promoter of islet survival in the manufacture of a composition useful for the enhancement of viability of islet cells as well as insulin secretion in vivo.

In some embodiments, a mammal is administered a therapeutically effective amount of a CC compound. The term “therapeutically effective amount” is used in this application to mean a dose that is effective in enhancing the viability and function of endocrine and exocrine pancreatic cells as well as other cell types affected by the diabetic state thereby improving blood glucose profile and reducing diabetic complications.

### DETAILED DESCRIPTION OF THE INVENTION

### I. Active Components

The compounds used in the application, collectively termed “CC compounds”, contain a heterocyclic moiety to which a quaternary ammonium-containing moiety is attached according to the following formula:

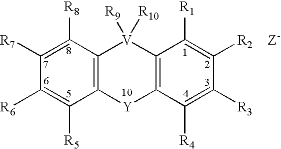

wherein R1 and R3-8 are independently hydrogen, C1-C26 straight, branched or cyclic alkanes or alkenes, aromatic hydrocarbons, alcohols, ethers, aldehydes, ketones, carboxylic acids, amines, amides, nitriles, or five- and/or six-membered heterocyclic moieties;
wherein R9 and R10 considered together are ═O or ═CH-L-N⁺(R11, R12, R13) or
wherein R9 and R10 considered independently are —OH or -L-N⁺(R11, R12, R13);

wherein R2 is represented by the formula: —X or —X′-L-N⁺(R11, R12, R13)Z⁻ or -L-N⁺(R11, R12, R13)Z⁻;

wherein V is —S—, —Se—, —C—, —O— or —N;

wherein Y is —S—, —Se—, —C—, —O— or —N;

wherein -L-N⁺(R11, R12, R13) can be linked to V or Y if V or Y is —N or can be linked to V and Y if V and Y are both —N;

wherein X is CH3 or Hydrogen;

wherein —X′ is —CH2-, —OCH2-, —CH2O—, —SCH2- or —CH2S—;

wherein L is a C1-C4 straight alkane, alkene, thiol, ether, or amine;

wherein R11, R12 and R13 are independently Hydrogen, C1-C4 straight alkanes, alkenes, thiols, amines, ethers or alcohols; and

wherein Z⁻ is Cl⁻, Br⁻ or I⁻.

In some embodiments, the quaternary ammonium containing moiety is choline ((2-hydroxyethyl)-trimethylammonium).

One embodiment of these compounds is [3-(3,4-Dimethyl-9-oxo-9H-thioxanthen-2-yloxy)-2-hydroxypropyl]trimethyl-ammonium chloride, or CCompound1. CCompound1 was acquired commercially (Sigma-Aldrich) or synthesized by a method indicated for other CC compounds below.

Two other embodiments of these compounds are N,N-diethyl-N-methyl-2-[(9-oxo-9H-thioxanthen-2-yl)methoxy]-ethanaminium iodide, or CCompound3, and N,N,N-trimethyl-3-(9H-thioxanthen-9-ylidene)-propane-1-aminium iodide, or CCompound19, all which were newly synthesized as reported in U.S. patent application Ser. No. 11,458,502; filed on Aug. 9, 2006; entitled “Compounds and compositions to control abnormal cell growth”; inventor: Zoltan Kiss, which is herein incorporated by reference in its entirety.

CCompound1, CCompound3 and CCompound19 were all found to prevent drastic increase in blood glucose level in STZ-treated animals. As such, CC compounds exert measurable protective effects on endocrine and exocrine pancreatic cells as well as other cells affected by a diabetic state.

An additional feature of the class of these heterocyclic compounds, as represented by CCompound1, CCompound19, and CCompound3 in this Application, is that they also exert anticancer effects [U.S. patent application Ser. No. 11,458,502; filed on Aug. 9, 2006; entitled “Compounds and compositions to control abnormal cell growth”; inventor: Zoltan Kiss]. Thus, while CC compounds protect normal panreatic cells, in subjects with cancer they would also simultaneously inhibit tumor growth. Accordingly, promotion of pancreatic cell viability by CC compounds does not extend to pancreatic cancer cells. This is further supported by data under “Examples” showing that concentrations of CCompound1 that promote survival and proliferation of stem cells reduce the number of pancreatic cancer cells.

TABLE 1
A Representative List of CCcompounds Used in the Invention.

| Trivial name | Chemical name | Structure |
|---|---|---|
| CCcompound 1 | [3-(3,4-Dimethyl-9-oxo-9H-thioxanthen-2-yloxy)-2-hydroxypropyl]trimethyl-ammonium chloride | 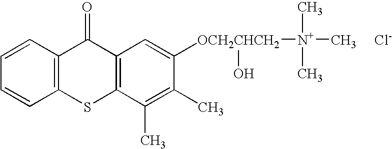 |
| CCcompound 2 | N,N,N-Trimethyl-2-[(9-oxo-9H-thioxanthen-2-yl)methoxy]-ethanaminium iodide | 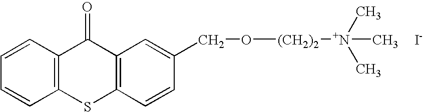 |
| CCcompound 3 | N,N-Diethyl-N-methyl-2-[9-oxo-9H-thioxanthen-2-yl)methoxy]-ethanaminium iodide | 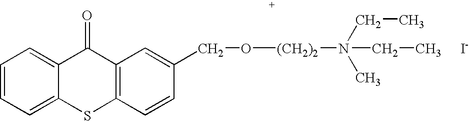 |
| CCcompound 4 | N,N,N-Triethyl-2-[(9-oxo-9H-thioxanthen-2-yl)methoxy]-ethanaminium iodide | 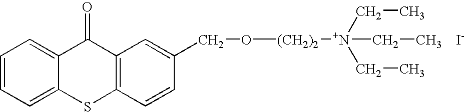 |
| CCcompound 5 | N,N-Ethyl-N,N-dimethyl-2-[9-oxo-9H-thioxanthen-2-yl)methoxy]-ethanaminium iodide | 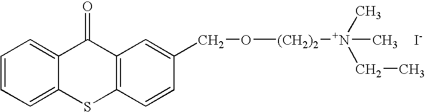 |
| CCcompound 6 | 2-{[2-(Diethylamino)ethoxy]methyl}-9H-thioxanthen-9-one hydrochloride | 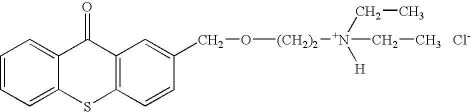 |
| CCcompound 7 | N,N,N-Trimethyl-2-[(9-oxo-9H-thioxanthen-2-yl)methoxy]-propan-1-aminium iodide | 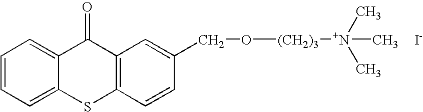 |
| CCcompound 8 | 2-{[2-(Dimethylamino)propoxy]methyl}-9H-thioxanthen-9-one hydrochloride | 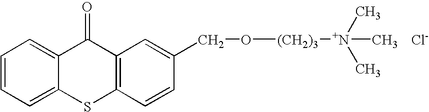 |
| CCcompound 9 | N,N,N-Triethyl-2-[(9-oxo-9H-thioxanthen-2-yl)methoxy]-propane-1-aminium iodide | 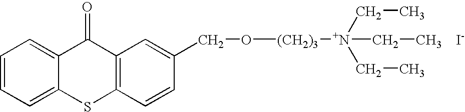 |
| CCcompound 10 | N,N,N-Diethyl-N-methyl-2-[(9-oxo-9H-thioxanthen-2-yl)methoxy]-propane-1-aminium iodide | 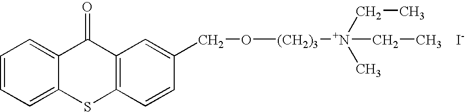 |
| CCcompound 11 | N,N,N-Dimethyl-N-ethyl-2-[(9-oxo-9H-thioxanthen-2-yl)methoxy]-propane-1-aminium iodide | 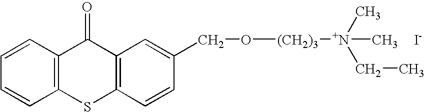 |
| CCcompound 12 | 2-{[3-(Diethylamino)propoxy]methyl}-9H-thioxanthen-9-one hydrochloride | 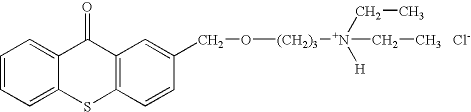 |
| CCcompound 13 | 2-Hydroxy-N,N-dimethyl-N-[(9-oxo-9H-thioxanthen-2-yl)methyl]-ethanaminium bromide | 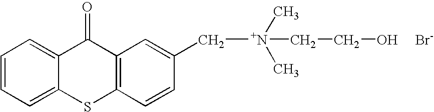 |
| CCcompound 14 | 2-Hydroxy-N,N-Diethyl-N-[(9-oxo-9H-thioxanthen-2-yl)methyl]-ethanaminium bromide | 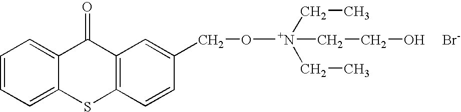 |
| CCcompound 15 | 3-Hydroxy-N,N-dimethyl-N-[(9-oxo-9H-thioxanthen-2-yl)methyl]propane-1-aminium bromide | 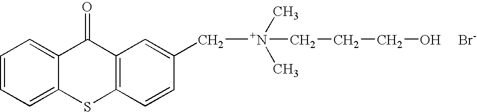 |
| CCcompound 16 | 3-Hydroxy-N,N-diethyl-N-[(9-oxo-9H-thioxanthen-2-yl)methyl]-propane-1-aminium bromide | 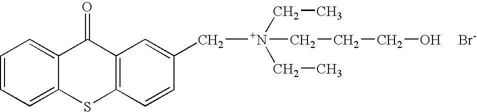 |
| CCcompound 17 | 3-(9-hydroxy-9H-thioxanthen-9-yl)-N,N,N-trimethyl-propane-1-aminium iodide | 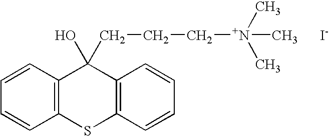 |
| CCcompound 18 | 3-(9-hydroxy-9H-selenoxanthen-9-yl)-N,N,N-trimethyl-propane-1-aminium iodide | 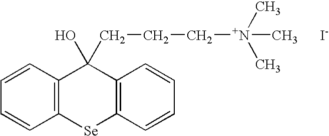 |
| CCcompound 19 | N,N,N-trimethyl-3-(9H-thioxanthen-9-ylidene)-propane-1-aminium iodide | 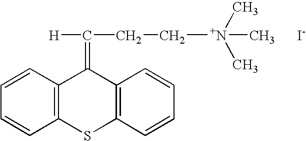 |
| CCcompound 20 | N,N,N-trimethyl-3-(9H-selenoxanthen-9-ylidene)-propane-1-aminium iodide | 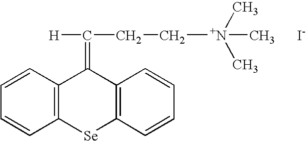 |
| CCcompound 21 | N,N,N-trimethyl-3-(2-methyl-9H-thioxanthen-9-ylidene)-propane-1-aminium iodide | 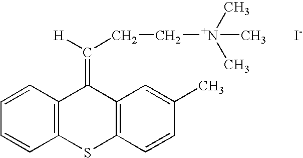 |
| CCcompound 22 | N,N-Dimethyl-N-ethyl-3-(2-methyl-9H-thioxanthen-9-ylidene)-propane-1-aminium iodide | 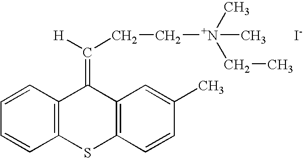 |
| CCcompound 23 | N,N-Diethyl-N-methyl-3-(2-methyl-9H-thioxanthen-9-ylidene)-propane-1-aminium iodide | 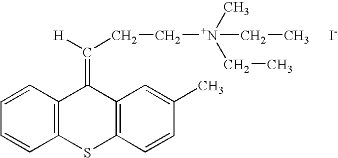 |
| CCcompound 24 | N,N-Dimethyl-N-allyl-3-(2-methyl-9H-thioxanthen-9-ylidene)-propane-1-aminium bromide | 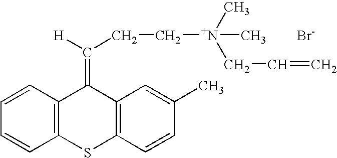 |
| CCcompound 25 | N,N,N-Triethyl-3-(2-methyl-9H-thioxanthen-9-ylidene)-propane-1-aminium iodidez | 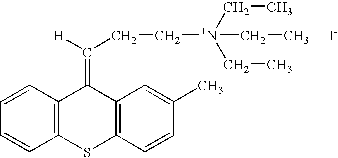 |
| CCcompound 26 | N,N-Diethyl-N-allyl-3-(2-methyl-9H-thioxanthen-9-ylidene)-propane-1-aminium bromide | 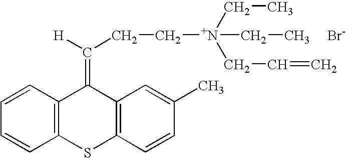 |

### II. Methods of Treatments.

CC compounds used in this invention are well soluble in dimethylsulfoxide and for all practical applications sufficiently soluble in water. Accordingly, oral application is one of the major administration routes to deliver a CC compound. In one embodiment of the invention, the CC compound is in the form of a tablet, gel capsule, a liquid, or the like. In each case, the CC compound is mixed with one or more carriers chosen by one having ordinary skill in the art to best suit the goal of treatment. In addition to the active compounds, the tablet or gel capsule may contain any component that is presently used in the pharmaceutical field to ensure firmness, stability, solubility and appropriate taste. In some embodiments, additional components of the tablet or gel will be chemically inert; i.e., it will not participate in a chemical reaction with the CC compound or the additives.

CC compounds may also be applied via intravenous, intraarterial, intraportal, intradermal, intraperitoneal, subcutaneous, intra-tissue or intramuscular delivery routes. In some embodiments, the CC compound may be delivered via infusion over a period of time or by using an osmotic minipump inserted under the skin for controlled release. The injectable solution may be prepared by dissolving or dispersing a suitable preparation of the CC compound in water or water-based carrier such as 0.9% NaCl (physiological saline) or phosphate buffered saline. Alternatively, the CC compound may dissolved first in dimethylsulfoxide and then diluted (100-400-fold dilution) in a physiologically compatible carrier using conventional methods. As an example only, a suitable composition comprises a CC compound in a 0.9% physiological saline solution to yield a total CC compound concentration of 0.1-g/ml or 25.0-g/ml.

A suitable dosage for oral or injection administration may be calculated in milligrams or grams of the active agent(s) per square meter of body surface area for the subject. In one embodiment, the therapeutically effective amount of CC compound is administered orally at a dose between 100-mg to 2,000-mg per m² body surface of the mammal. In another embodiment, the CC compound is administered by an injection method at a dose of 50-mg to 1,000-mg per m² body surface of the mammal.

The amount of the CC compound may vary depending on the method of application. For example, in case of intravenous application the required amount may approach the lower limit, while in case of subcutaneous application the required amount may be closer to the upper limit.

Application of the CC compound orally or by one of the above injection application methods may be repeated as many times as needed to achieve a satisfactory reduction in the level of islet cell death. However, for practical reasons, oral administration can be made more frequent than injection applications.

In one embodiment, the therapeutically effective amount of CC compound may be administered once daily. In another embodiment, the dose is administered twice or three times daily. In still another embodiment, administration of the CC compound is performed three-times a week. In some embodiments, the example dosage amounts provided above are given once daily, or less frequently than once daily (e.g., every other day or three times a week). In other embodiments, if application is repeated several times a day, the dosages may be lowered compared to the amounts provided above.

An important decision that the health care provider needs to make concerns the start and length of treatment with the CC compound. Since CC compound protects against β-cell damage at any time during the development of diabetes, administration may begin as soon as deterioration in islet cell function is noted. In case of pancreatitis and pancreatic cancer, the treatment may be started immediately after diagnosis. Administration of CC compound may be needed over the entire remaining life time or may be restricted to a time period when there is evidence that decline in the number of viable β-cells has been sufficiently reduced, stopped, or reversed.

The CC compound may be used together with insulin or any other anti-diabetic medication or medication used to alleviate the symptoms of pancreatitis or treat pancreatic cancer. The CC compound may also be used to treat patients after pancreatectomy, usually resulting in the removal of 80-90% of pancreas, to preserve the function of remaining endocrine and exocrine cells. Another application of the CC compound is to prevent β-cell death following islet transplantation into type 1 diabetic patients or patients treated with pancreatectomy. The selected CC compound may also be used together with other agents, or enhancers, that positively influence(s) the proliferation of progenitors of islet cells and the survival of differentiated islet β-cells. Examples for such agents include incretin glucagon-like peptide analogues, α1-antitrypsin, placental alkaline phosphatase, pioglitazone or a related thiazolidinedione compound, cytokines and growth factors such as insulin, insulin like growth factor-1, growth hormone, platelet-derived growth factor, fibroblasts growth factor, placental growth factor, epidermal growth factor, vascular endothelial growth factor, transforming growth factors as well as testosterone and amino acids such as leucine, lysine or arginine.

In case of oral administration of the CC compound, the enhancer(s) may be applied together with, or separately from, the CC compound. In case of injection application, the enhancer(s) and the CC compound may be dissolved or suspended in the same physiologically compatible carrier, or they can be applied separately.

In a further embodiment, the invention provides for the use of a CC compound for the protection of islet cells during isolation, during transplantation, and after transplantation. For in vitro protection of islet cells, in some embodiments the medium comprises 2-15 μM of a CC compound. In other embodiments, the medium comprises about 2-10 μM of a CC compound.

Islet cells may be transplanted together with adult stem cells, such as MSCs and hemopoietic stem cells that cooperatively enhance viability of islet cells. In some embodiments, in preparation for transplantation of such a mixed cell population, stem cell cultures are treated with 2-15 μM of a CC compound for a time period required for achieving optimal cell numbers and viability. In other embodiments, stem cell cultures are treated with 2-10 μM of a CC compound for a time period required for achieving optimal cell numbers and viability. In some embodiments, a CC compound would be present in the transplant suspension. As an alternative, or in addition to, treating a cell suspension with CC compounds, after transplantation of mixed cell population, a CC compound may be administered to the host orally or via injection at a frequency and concentration required for optimal survival of transplanted cells as discussed above.

In an additional embodiment, stem cells and a CC compound may be simultaneously transplanted without islet cells to a subject with diabetes to enhance viability of endogenous islet cells. This may be followed by additional oral or injection treatment with the CC compound.

Finally, in an additional embodiment, the invention provides for the use of a CC compound alone or together with another promoter of cell survival in the manufacture of a composition useful for the enhancement of viability and function of endocrine and exocrine pancreatic cells, stem cells as well as other cell types affected by the diabetic state in vivo.

### EXAMPLES

### Example 1

### Use of the MTT Assay to Determine Cell Viability

In the Examples below, an MTT assay was used to determine the relative number of viable cells after treatments. This calorimetric assay is based on the ability of living cells, but not dead cells, to reduce 3-(4,5-dimethyl thiazol-2-yl)-2,5-diphenyltetrazolium bromide. [Carmichael, J, De Graff, W. G., Gazdar, A. F., Minna, J. D. and Mitchell, J. B. (1987) Evaluation of tetrazolium-based semiautomated calorimetric assay: Assessment of chemosensitivity testing. Cancer Res. 47, 936-942], which is hereby incorporated by reference. For this assay, cells were plated in 96-well plates, and the MTT assay was performed as described in the above article both in untreated and treated cell cultures. The MTT assay also was performed at the time when the treatment was started to allow for assessment of the proliferation and survival rates in the control and treated cell cultures. Absorption was measured at wavelength=540, indicated in the Tables below as A₅₄₀. In the MTT assay, higher values mean proportionally higher numbers of viable cells.

### Example 2

### CCompound1 Enhances Viability of Streptozotocin (STZ)-Treated Islet β-Cells

NIT-1 β-cells, originally isolated from transgenic NOD mouse carrying SV 40 large T antigen gene on a rat insulin promoter, were obtained from American Type Culture Collection (ATCC CRL-2055). NIT-1 cells contain and secrete insulin, while at the same time they retained their ability to proliferate in the presence of an appropriate stimulus. The cells, maintained in Ham's F12K medium containing 10% heat-inactivated dialyzed fetal bovine serum, were used between passages 32-35.

STZ causes specific islet β-cell damage via the release of reactive oxygen species (ROS) and/or nitric oxide [Chen, H., Carlson, E. C., Pellet, L., Moritz, J. T. and Epstein, P. N. (2001) Overexpression of metallothionein in pancreatic β-cells reduces streptozotocin-induced DNA damage and diabetes. Diabetes 50, 2040-2046; Szkudelski, T. (2001) The mechanism of alloxan and streptozotocin action in B cells of the rat pancreas. Physiol. Rev. 50, 536-546]. Thus, in many respects, STZ's action involves the same pathological mechanisms that mediate the islet cell death-inducing effects of high glucose, saturated fatty acids, and autoimmune reactions. For this reason, STZ is a frequently used experimental tool to induce the death of β-cells. Accordingly, this invention uses STZ to elicit the death of β-cells both in vitro (see next) and in vivo (see later) as well as CC compounds in an attempt to provide protection.

In this experiment, NIT-1 cells were seeded into 96-well plates. After 24 hours, the medium was replaced with fresh 2% fetal bovine serum containing medium, followed by treatments with 2-50 μM of CCompound1 and 30 min later with 20 mM STZ. Treatments were for 30 hours followed by the MTT assay to determine the relative number of viable cells (expressed as A₅₄₀). The mean value ±std. dev. of 8 determinations (in 8 separate wells) for each treatment is shown in TABLE 2.

The data indicate that CCompound1 at 2-5 μM concentrations partially reverses the inhibitory effects of STZ on cell proliferation, while at a 10 μM concentration CCompound1 almost fully reverses the inhibitory effects of STZ on cell proliferation. In contrast, 50 μM of CCompound1 added to the inhibitory effects of STZ.

TABLE 2
Low concentrations of CCcompound1 protect β-cells against
STZ-induced cell death in vitro.

| Additions | A₅₄₀ |
|---|---|
| 0 time | 0.987 ± 0.108 |
| 28 h control | 1.210 ± 0.047 |
| STZ, 20 mM | 0.764 ± 0.051 |
| STZ + CCcompound1, 2 μM | 0.934 ± 0.053 |
| STZ + CCcompound1, 5 μM | 1.078 ± 0.068 |
| STZ + CCcompound1, 10 μM | 1.144 ± 0.056 |
| STZ + CCcompound1, 50 μM | 0.718 ± 0.071 |

### Example 3

### Protective Effect of CCompound1 against Fatty Acid-Induced Death of RINm5F Islet β-Cells In Vitro

Saturated fatty acids (palmitic acid or stearic acid) induce apoptotic cell death of RIN 1046-38 cells [Eitel, K., Staiger, H., Rieger, J., Mischak, H., Brandhorst, H., Brendel, M. D., Bretzel, R. G., Haring, H. U. and Kellerer, M. (2003) Protein kinase C δ activation and translocation to the nucleus are required for fatty acid-induced apoptosis of insulin-secreting cells. Diabetes 52, 991-997]. Since fatty acids contribute to β-cell loss in vivo, protection of these cells against fatty acid-induced death can improve the condition of in diabetic subjects.

RINm5F rat islet β-cells (ATCC CRL-2058; secondary clone of RIN-m clone secreting only insulin, but no somatostatin or glucagon) were used to determine if CCompound1 could protect them against fatty acid-induced cell death. The medium for propagation: RPMI 1640/10% fetal bovine serum.

Palmitic acid (purchased from Sigma-Aldrich) was dissolved in 200 mM ethanol and then diluted 1:25 with Krebs-Ringer-Hepes buffer containing 20% bovine serum albumin (fraction V, fatty acid-free; from Sigma-Aldrich). The fatty acid mixture was gently agitated at 37° C. under nitrogen overnight. The cells were seeded into 96-well plates at a density of 8,000 cells per well. When the cultures reached 70% confluence, fatty acid or a corresponding amount of albumin and CCompound1 (suspended in the medium) were added to the medium and incubations continued for 24 hours. This was followed by the MTT assay to determine the relative number of viable cells.

As shown in the TABLE 3, 0.5 mM palmitic acid decreased the number of viable cells by 28% compared to the untreated incubated control. CCompound1 at 5 μM partially prevented, while at 10 μM concentration almost fully prevented, palmitic acid-induced decrease in the number of viable cells. In contrast, 50 μM CCompound1 added to the inhibitory effect of palmitic acid.

TABLE 3
CCcompound1 (CC1) reduces fatty acid-induced death of
RINm5F 8 cells in vitro.
The data are expressed as mean values ± S.D. of 8 determinations.

| Addition | A₅₄₀ |
|---|---|
| None | 1,327 ± 0.081 |
| Palmitic acid, 0.5 mM | 0.950 ± 0.049 |
| Palmitic acid, 0.5 mM + CC1, 5 μM | 1,198 ± 0.086 |
| Palmitic acid, 0.5 mM + CC1, 10 μM | 1,251 ± 0.069 |
| Palmitic acid, 0.5 mM + CC1, 50 μM | 0.836 ± 0.080 |

### Example 4

### CCompound1 at Low Concentration Promotes Survival and Proliferation of Human Bone Marrow-Derived Mesenchymal Stem Cells (MSC) In Vitro

### Isolation and Maintenance of Human Bone Marrow-Derived Mesenchymal Stem Cells

Bone marrow aspirates were taken from normal adult donors signed after informed consent according to a protocol approved by the appropriate Ethics Committee. For the preparation of bone marrow mesenchymal stem cells, essentially a widely used technique was used as described earlier by others [Pittenger, M. F., Mackay, A. M., Beck, S. C., Jaiswal, R. K., Douglas, R., Mosca, J. D., Moorman, M. A., Simonetti, D. W., Craig, S, and Marshak, D. R., Multilineage potential of adult human mesenchymal stem cells (1999) Science 284, 143-147]. Briefly, nucleated cells were isolated with a pre-prepared commercial density gradient (Lymphoprep, Nycomed, Pharma, Oslo, Norway) and resuspended in Dulbecco's modified Eagle's medium (DMEM) (GIBCO, Grand Island, N.Y.) supplemented with 10% fetal calf serum (FCS), 50 U/ml of penicillin, and 50 μg/ml of streptomycin (GIBCO). All nucleated cells were plated in 25-cm² flasks (BD Falcon, Bedford, Mass.) at 37° C. in humidified atmosphere containing 5% CO₂. After 24 hours, nonadherent cells were removed and cryopreserved in liquid nitrogen until use. The remaining adherent cells were thoroughly washed with Hanks balanced salt solution (HBSS) (GIBCO). Fresh complete culture medium was added and replaced every 3 or 4 days (twice a week). When cells grew to about 80% confluence, they were suspended and harvested by incubating with a ready-made solution containing 0.25% trypsin and 1 mM EDTA (Sigma-Aldrich, St. Louis, Mo.) for 5 minutes at 37° C.; this cell suspension is designated as passage 1. These cells were further expanded with 1:3-1:5 splitting in 175-cm² flasks (BD Falcon).

The total numbers of nucleated and viable cells were determined with a hemocytometer, using Turck's solution and trypan blue stain, respectively. The morphology of MSC was examined every week under an inverted microscope (Olympos CK2, Tokyo, Japan) to verify that cells retained their structural characteristics. In the two experiments described below, MSCs were split into 24-well “F-bottom” plates at passage 5.

The results from the first and second experiment are shown in TABLE 4 and 5 respectively. After plating, only a relatively small fraction of MSCs remained attached to the plate's surface after 24 hours; non-attached (probably less viable) cells were removed during subsequent medium change. Once the cells attached, CCompound1 at 5-10 μM concentrations appeared to slightly enhance cell numbers both in the absence or presence of 2% serum during the first 3 days. Importantly, both in the absence and presence of 2% serum, 5-10 μM concentrations of CCompound1 significantly increased the expansion of stem cells between days 3 and 6. In fact, 10 μM CCompound1 enhanced cell proliferation even in the presence of 10% serum.

It is critical that soon after medium change the cells are treated with the CC compound. In one experiment when CCompound1 was added to cells 6 hours after medium change, the effects on cell survival and proliferation were 50-70% smaller (data not shown).

It should be stated that in these experiments the effects of CCompound1 on stem cell survival (i.e. its effect on viability and attachment during the first 24 hours after plating) remained unknown. This issue was addressed in an experiment described below.

### TABLE 4 and TABLE 5. Lower Concentrations of CCompound1 (CC1) Promote Expansion of Human Mesenchymal Stem Cells.

In both experiments, about 80% confluent cells were split into 24-well “F-bottom” plates. Then after 24 hours in 10% serum containing medium (at about 30-35% confluence), the medium was replaced for either serum-free medium or 2% or 10 fetal calf serum containing medium. The cells were then immediately treated with 2-100 μM concentrations of CCompound1 as indicated in the Tables. Then incubations were continued in the absence or presence of 0, 2 or 10% serum, as indicated, for 3-6 days. In both experiments, the data are the mean ±S.D. of 5 incubations.

TABLE 4

| Incubation time | | CC1 | CC1 | CC1 |
|---|---|---|---|---|
| (day) | None | 5 μM | 10 μM | 50 μM |
| | Cell number per culture (×10⁻³) | | | |
| | 0% serum | | | |
| 0 | 20 | 20 | 20 | 20 |
| 2 | 4.2 ± 1.3 | 6.4 ± 2.0 | 5.9 ± 1.9 | 4.0 ± 1.6 |
| 4 | 4.7 ± 2.2 | 8.1 ± 2.1 | 6.8 ± 2.4 | 3.4 ± 1.1 |
| 6 | 5.1 ± 2.0 | 12.5 ± 1.6* | 10.2 ± 1.4* | 3.8 ± 1.2 |
| | 2% serum | | | |
| 0 | 20 | 20 | 20 | 20 |
| 2 | 4.7 ± 1.6 | 7.1 ± 2.7 | 6.3 ± 1.6 | 3.9 ± 1.1 |
| 4 | 7.1 ± 1.9 | 11.6 ± 2.4* | 9.2 ± 2.3 | 5.2 ± 1.6 |
| 6 | 11.8 ± 3.0 | 20.8 ± 2.5* | 17.4 ± 2.0* | 7.0 ± 3.3 |

*P < 0.05

TABLE 5

| | Cell number/culture (×10⁻³) | | | | | | | | |
|---|---|---|---|---|---|---|---|---|---|
| | 0% serum | | | 2% serum | | | 10% serum | | |
| CC1 (μM) | Day 0 | Day 3 | Day 6 | Day 0 | Day 3 | Day 6 | Day 0 | Day 3 | Day 6 |
| 0 | 20 | 4.1 ± 2.9 | 5.5 ± 2.6 | 20 | 4.4 ± 1.9 | 11.3 ± 3.7 | 20 | 5.2 ± 1.3 | 31.5 ± 4.1 |
| 2.0 | 20 | 4.7 ± 3.5 | 6.2 ± 1.7 | 20 | 4.9 ± 2.6 | 16.4 ± 2.5* | 20 | 5.9 ± 1.7 | 33.3 ± 5.2 |
| 5.0 | 20 | 5.9 ± 2.1 | 10.3 ± 2.5* | 20 | 7.2 ± 1.3 | 18.3 ± 4.8* | 20 | 6.2 ± 2.6 | 36.4 ± 5.2 |
| 10.0 | 20 | 6.3 ± 2.2 | 13.3 ± 3.2* | 20 | 5.6 ± 1.6 | 19.0 ± 3.8* | 20 | 5.1 ± 1.7 | 39.1 ± 4.1* |
| 25.0 | 20 | 4.4 ± 1.7 | 6.4 ± 3.1 | 20 | 4.7 ± 2.6 | 12.9 ± 4.9 | 20 | 5.2 ± 2.1 | 33.5 ± 5.3 |
| 100.0 | 20 | 1.1 ± 1.6 | 1.3 ± 1.1 | 20 | 1.5 ± 1.1 | 5.8 ± 3.2 | 20 | 4.4 ± 2.5 | 14.8 ± 3.1 |

*P < 0.05

Next, CCompound1 was present during plating and cells were allowed to grow in the presence of 10% serum or 2% serum for 24 hours followed by first removing non-attached cells and then counting the cells. As shown in TABLE 6, in the presence of 10% serum more cells attached to the surface than in the presence of 2% serum. In both cases, 5-10 μM CCompound1 enhanced the number of attached cells. Since attachment probably relates to the viability of cells, the experiment shows that CCompound1 is able to increase the survival of stem cells as well.

TABLE 6
Low concentration of CCcompound1 (CC1) promotes survival
of human mesenchymal stem cells.
Cells were incubated in the presence of 5, 10, and 25 μM
concentrations of CCcompound1 in the presence of 10% serum (A)
or 2% serum (B) for 24 hours. The data are the mean ± S.D. of
5 incubations.

| | | CC1 | CC1 | CC1 |
|---|---|---|---|---|
| Incubation time | None | 5 μM | 10 μM | 25 μM |
| | Cell number per culture (×10⁻³); 10% serum A | | | |
| 0 h | 20 | 20 | 20 | 20 |
| 24 h | 4.8 ± 1.7 | 8.9 ± 2.8* | 10.9 ± 3.2* | 3.8 ± 1.9 |
| | Cell number per culture (×10⁻³); 2% serum B | | | |
| 0 h | 20 | 20 | 20 | 20 |
| 24 h | 2.2 ± 1.3 | 6.4 ± 2.4* | 7.9 ± 2.6* | 2.4 ± 1.4 |

*P < 0.05

### Example 6

### Effects of CCompound1, CCompound3, and CCompound 19 on Blood Glucose Level and Body Weight in STZ-Treated Mice

Male C57BL/6 mice (12 weeks old) were housed in a specific pathogen-free facility with a 12-h light/dark cycle and given free access to food and water. Animals weighing between 28-31 g were selected for the experiments. In each group, at the start of experiment the body weight among individual animals differed by less than 1-g.

One group of mice received no treatment over the entire experiment. A second group received only 4.5 mg per kg of CCompound1 on days −2, −1, 0, +1, +2, +3, +4, +5, +6 and +7. A third/fourth/fifth/sixth group of animals were treated i.p. with 210 mg/kg of STZ on day 0. The fourth, fifth, and sixth group of animals also received 4.5 mg per kg each of CCompound1 (CC1; 4th group) or CCompound3 (CC3; 5th group), or CCompound19 (CC19; 6th group) on days −2, −1, 0, +1, +2, +3, +4, +5, +6 and +7 (with STZ treatment given on day 0). Each group consisted of 7 animals. Body weight was measured regularly.

CC compounds were dissolved in physiological saline while STZ was dissolved in 100 mM sodium citrate (pH 4.5). All compounds were injected intraperitoneally in 0.5 ml volume. Control animals were injected physiological saline (0.5 ml) on the same days when other groups were treated with CC compounds.

Blood samples were taken from the eye (canthus) of the animals on days −1, 0, +1, +2, +3, +7 and +10, and glucose concentrations in whole blood samples were immediately measured with the commercially available fast Glucose C test. The animals were not fasted prior to taking the blood samples. The results are expressed as the mean values from 7 animals ±std. dev. (S.D.).

The results, shown in TABLE 7, indicate that CCompound1 alone had no effect on blood glucose level; the same is expected of CCompound3 and CCompound19. STZ treatment increased blood glucose level about 4-fold by day 3; after that the blood glucose remained steady. Each CC compound significantly reduced the effect of STZ on blood glucose. These data indicate that CC compounds are capable of protecting the insulin producing islet β-cells.

TABLE 7
CCcompound1 (CC1), CCcompound19 (CC19), and
CCcompound3 (CC3) partially protect islet cells against STZ action.

| | | | | | | |
|---|---|---|---|---|---|---|
| Treatment | | | | STZ + | STZ + | STZ + |
| Day | None | CC1 | STZ | CC1 | CC3 | CC19 |

Blood glucose (mM)

| | | | | | | |
|---|---|---|---|---|---|---|
| −1 | 4.7 ± 0.6 | 4.5 ± 0.4 | 4.8 ± 0.8 | 4.6 ± 0.7 | 4.6 ± 0.5 | 4.3 ± 0.3 |
| 0 | 4.5 ± 0.6 | 4.5 ± 0.4 | 4.8 ± 0.6 | 4.1 ± 0.4 | 4.7 ± 0.7 | 4.8 ± 0.3 |
| +1 | 4.4 ± 0.5 | 4.7 ± 0.6 | 8.5 ± 1.6 | 5.7 ± 0.5 | 6.5 ± 0.7 | 5.9 ± 0.6 |
| +2 | 4.8 ± 0.5 | 4.6 ± 0.4 | 14.9 ± 1.5 | 8.2 ± 1.4 | 10.7 ± 1.1 | 8.7 ± 0.8 |
| +3 | 5.0 ± 0.5 | 4.7 ± 0.4 | 19.0 ± 1.4 | 10.1 ± 1.5 | 13.0 ± 1.1 | 10.5 ± 0.9 |
| +7 | 4.9 ± 0.7 | 4.6 ± 0.4 | 19.8 ± 1.6 | 10.9 ± 1.2 | 14.2 ± 1.3 | 12.5 ± 1.1 |
| +10 | 4.5 ± 0.8 | 4.3 ± 0.6 | 18.9 ± 1.3 | 11.0 ± 0.8 | 13.6 ± 1.6 | 11.9 ± 1.1 |

In the same experiment described in TABLE 7, STZ reduced body weight by about 30% by day 10. CCompound1, CCompound3, and CCompound19 each reduced the effect of STZ on body weight; these data are shown in TABLE 8. Since insulin plays a key role in the maintenance of body weight, these data confirm that CC compounds promote sufficient secretion of insulin by surviving islets to prevent a major decline in body weight.

TABLE 8
CCcompound1 (CC1), CCcompound19 (CC19) and CCcompound3 (CC3)
partially prevent the body weight-reducing effect of STZ.

| | | | | STZ + | STZ + | STZ + |
|---|---|---|---|---|---|---|
| Treatment Day | None | CC1 | STZ | CC1 | CC3 | CC19 |
| 0 | 28.5 ± 1.0 | 28.2 ± 0.9 | 29.7 ± 1.2 | 28.2 ± 0.7 | 28.0 ± 0.9 | 28.1 ± 0.5 |
| +1 | 29.1 ± 1.1 | 28.7 ± 0.4 | 28.1 ± 0.9 | 28.0 ± 0.7 | 28.2 ± 0.8 | 28.5 ± 1.2 |
| +2 | 29.5 ± 0.7 | 28.1 ± 0.4 | 24.4 ± 1.3 | 26.9 ± 0.8 | 25.3 ± 0.5 | 26.4 ± 0.8 |
| +3 | 29.8 ± 0.9 | 28.7 ± 0.7 | 23.4 ± 1.5 | 26.9 ± 0.5 | 25.3 ± 0.6 | 26.8 ± 1.0 |
| +7 | 31.2 ± 1.4 | 29.5 ± 0.8 | 22.1 ± 0.9 | 27.1 ± 0.7 | 25.2 ± 0.9 | 26.2 ± 0.9 |
| +10 | 31.6 ± 0.8 | 30.3 ± 1.4 | 21.1 ± 1.5 | 27.2 ± 0.9 | 24.7 ± 1.3 | 26.4 ± 1.7 |

In a second similar experiment performed with 6 mice in each group, the changes in body weight were followed for up to 80 days (when the last STZ-treated mouse died), and survival was followed for up to 100 days. In this second experiment, STZ, STZ+CCompound1 and STZ+CCompound3 had similar effects on blood glucose level as presented in TABLE 7.

For the body weight (on the 80th day) and survival the following values were obtained: Untreated (6 survivors), 37.7 g; STZ-treated (one last survivor died on day 80, others survived for 22, 25, 27, 60, 75 days), 26.0 g; CCompound1-treated (6 survivors), 36.0 g; STZ+CCompound1 treated (6 survivors), 33.4 g; STZ+CCompound3-treated (5 survivors; one mouse died on day 78), 32.5 g. At the start of the experiment (day 0), the average body weight ranged from 28.6 to 29.8 g. Accordingly, by day 80, STZ-treated animals that were also treated with CCompound1 and CCompound3 gained about 50% of the weight gained by the untreated animals.

These data confirm a correlation between the effects of compounds on blood glucose (i.e. islet survival), body weight, and survival. In STZ-treated mice, CC compounds reduce blood glucose, prevent body weight loss and promote survival.

The effects of CCompound1 on blood glucose level and body weight as well as survival were also examined in a third experiment. Animals weighing between 24.6-25.3 g were selected for this experiment with each group consisting of 6 mice. One group of mice (group 1) received no treatment over the entire experiment, while in the other two groups (groups 2-3), animals were injected (i.p.) with 210 mg/kg of STZ on day 0. Animals in group 3 were also injected (i.p.) with 4.5 mg per kg of CCompound1 once daily for 10 days starting 24 h prior to STZ treatment. CCompound1 and STZ were dissolved as previously indicated.

In this experiment the animals were fasted for 13-14 hours (fasted overnight from 6 p.m. to 7-8 a.m. and returned to normal diet from 8 a.m. to 6 p.m) prior to taking the blood samples. While such starvation regimen resulted in low blood glucose levels in the control group, it also reduced intra-group variations in the blood glucose values.

The results in TABLE 9 show that on days 3 and 4, CCompound1 reduced the effects of STZ on blood glucose level by more than 50%. Although treatments with CCompound1 were stopped on day 9, in the corresponding group there was no significant elevation in the blood glucose levels between days 9 and 21. These data again indicate that islet cell destruction in STZ+CCompound1-treated animals reached significantly lower levels compared to animals treated with STZ alone, and that in this group islet cell death did not continue after the 9th day.

As shown in TABLE 10, STZ treatment decreased body weight between day 0 and day 21 by 35% (8.7 g). In contrast, animals co-treated with CCompound1 and STZ lost only 0.7-1.2 g body weight. It is noteworthy, that although treatments with CCompound1 stopped on day 9, the condition of animals remained stable during the subsequent 12 days period.

In this experiment, animals were sacrificed on day 53. On that day only 2 mice were alive in the STZ-treated group (with the others died on day 32, 35, 39 and 46), while all 6 mice were alive in the group treated with STZ+CCompound1. As in the previous experiment, CCompound1 greatly promoted the survival of STZ-treated animals.

TABLE 9
Effects of CC1 on blood glucose level in STZ-treated mice.

| | Blood glucose (mM) n = 6 | | |
|---|---|---|---|
| Day | None | STZ | STZ + CC1 |
| −1 | 1.9 ± 0.3 | 1.9 ± 0.3 | 2.1 ± 0.3 |
| 0 | 2.2 ± 0.4 | 2.3 ± 0.4 | 2.1 ± 0.2 |
| 1 | 2.2 ± 0.3 | 10.7 ± 0.9 | 6.7 ± 0.7 |
| 2 | 2.3 ± 0.3 | 14.4 ± 1.5 | 7.1 ± 0.5 |
| 3 | 2.2 ± 0.5 | 16.2 ± 1.3 | 7.2 ± 0.5 |
| 4 | 1.8 ± 0.5 | 17.1 ± 0.8 | 7.6 ± 0.4 |
| 5 | 1.9 ± 0.4 | 17.4 ± 1.1 | 8.2 ± 0.4 |
| 6 | 1.9 ± 0.6 | 16.9 ± 1.2 | 8.6 ± 0.3 |
| 7 | 1.8 ± 0.5 | 17.4 ± 2.4 | 9.3 ± 0.5 |
| 8 | 2.0 ± 0.5 | 16.7 ± 2.6 | 10.4 ± 0.7 |
| 9 | 2.3 ± 0.4 | 18.1 ± 1.3 | 11.0 ± 1.1 |
| 14 | 2.5 ± 0.3 | 17.5 ± 1.2 | 11.8 ± 1.0 |
| 21 | 2.1 ± 0.3 | 18.4 ± 0.7 | 12.5 ± 0.8 |

TABLE 10
Effects of CC1 on the body weight of STZ-treated mice.

| | Body weight (g) n = 6 | | |
|---|---|---|---|
| Day | None | STZ | STZ + CC1 |
| 0 | 24.8 ± 0.5 | 25.3 ± 0.4 | 25.1 ± 0.5 |
| 3 | 25.1 ± 0.7 | 22.5 ± 0.8 | 24.3 ± 1.0 |
| 5 | 25.4 ± 0.8 | 20.3 ± 0.9 | 24.2 ± 0.9 |
| 7 | 26.0 ± 0.7 | 19.7 ± 0.6 | 24.1 ± 0.7 |
| 9 | 26.7 ± 0.6 | 19.0 ± 0.7 | 24.3 ± 0.8 |
| 11 | 27.2 ± 0.6 | 18.7 ± 0.9 | 24.3 ± 0.9 |
| 13 | 27.9 ± 0.8 | 18.6 ± 1.0 | 24.5 ± 1.1 |
| 15 | 28.6 ± 0.9 | 18.1 ± 0.8 | 23.9 ± 0.4 |
| 17 | 29.0 ± 1.3 | 17.5 ± 1.1 | 24.5 ± 1.1 |
| 21 | 29.8 ± 1.3 | 16.6 ± 1.2 | 24.4 ± 1.0 |

### Example 7

### Effects of Ccompound1 on Blood Glucose Level as Well as Damage of the Exocrine Pancreatic Tissue in the L-Arginine-Treated Rat Model of Necrotic Pancreatitis

L-arginine-treated rats are frequently used experimental tools to model necrotic pancreatitis [Krajewski, E., Krajewski, J., Spodnik, J. H., Figarski, A. and Kunasik-Juraniec, J. (2005) Changes in the morphology of the acinar cells of the rat pancreas in the oedematous and nectoric types of experimental acute pancreatitis. Folia Morphol. 64, 292-303]. L-arginine is a precursor of NO which primarily destroys the exocrine pancreas with subsequent damage of insulin producing islet cells. Just like pancreatitis is often associated with elevated blood glucose level in human patients, extensive damage of pancreas in the L-arginine-treated animal model also results in damaged function of β-cells as indicated by higher blood glucose levels. In this example, the L-arginine-treated rat model was employed to see if CCompound1 could also prevent or reduce deterioration of β-cell function and the damage of the exocrine pancreas.

Male Wistar rats weighing 220-250 g were divided into four groups, each group consisting of 6 animals. To ensure standard conditions animals were starved for 16 hours before the start of the experiment. CCompound1 was first injected 24 hours prior to L-arginine injection, followed by similar daily injections for 5 consecutive days. In the first group, rats remained untreated. On day 0, animals in groups 2-4 were injected 2×2.5 g/kg L-arginine i.p. at one hour intervals. Animals in group 4 also received daily s.c. injection of 4.5 mg/kg of CCompound1 between days −1 and 5. To verify development of acute pancreatitis, 24 hours after L-arginine injections rats in group 2 were anesthetized with 45 mg/kg pentobarbital i.p. and exsanguinated through the abdominal aorta. On day 0, 2, 4 and 6 blood sugar values of the experimental animals were measured after fasting for 8 hours with the C-test; blood samples were taken from the tail. Edema of the pancreatic tissue was assessed by the pancreatic weight/body weight ratio. The animals in the remaining groups were sacrificed on day 7.

The body weight of experimental animals was measured at the beginning and the end of the experiment. The difference of the two values correlates with the development and severity of pancreatitis. Rats with severe pancreatitis did not gain weight, whereas in control animals body weight increased steadily during the experiment.

To examine if CCompound1 also had effects on areas of pancreas other than islets, the following determinations were made:

The pancreatic weight/body weight ratio (pw/bw) was evaluated as an estimate of the degree of pancreatic edema.

For histological examinations, a portion of the head of the pancreas was fixed in 4% neutral formaldehyde solution and embedded in paraffin. Tissue slices were stained with hematoxylin and eosin and examined by light microscopy. Slices were coded and examined blind by a pathologist for the grading of histological alterations. The extent and intensity of pancreatic edema, leukocyte infiltration, acinar vacuolization, hyperemia and tubular transformation were described with scores ranging from 0 to 3. The total histological damage was calculated by adding the scores for the various below listed parameters together.

Hyperemia; defined as the amount of red blood cells in the vessels.

Edema; defined as the widening of interstitial space.

Vacuolization; defined as the formation of vacuoles in the cytoplasm of acinar cells. Vacuolization results from the fusion of zymogene granules and lysosomes.

Inflammation; defined and quantified by the infiltration of the tissue with mononuclear inflammatory cells (lymphocytes, plasma cells, and mast cells).

Necrosis; defined as the measure of the extent of the destruction of normal pancreatic tissue structure.

Tubular transformation; defined as desquamation of acinar cell apical cytoplasm and release of cytoplasm segments into the acinar lumen leading to formation of duct-like tubular complexes. (Elsässer H. P., Adler G and Kern H. F. (1986) Time course and cellular source of pancreatic regeneration following acute pancreatitis in the rat. Pancreas 1 (5) 421-9).

Total histological damage (total damage); defined and calculated by combining the histological scores of the above mentioned parameters (except vacuolization). This value is the generally accepted best representation of the severity of pancreatitis in the art.

Determination of islet insulin content by immunohistochemistry: Immunohistochemical analysis of the expression of insulin was performed on 4% buffered formalin-fixed sections of the pancreas embedded in paraffin. The 4-μm-thick sections were stained with an automated system (Autostain; Dako, Glostrup, Denmark) and immunostaining was detected with EnVision detection system. Anti-insulin antibody was purchased from Histopathology Ltd, Hungary. Twenty high power fields were examined (blind) by a pathologist and the extent and intensity of insulin staining was counted. Slices were graded on a scale ranging from 0 to 3, where 0 value meant the absence of insulin positive cells whereas in the case of values 1, 2 and 3 the intensity of staining was between 0-33%, 33-66% and 66-99%, respectively.

Statistical analysis: Serum glucose values were compared by repeated measures for ANOVA and ANOVA analysis, using a Scheffe post hoc analysis. P values lower than 0.05 were considered statistically different.

As shown in TABLE 11, in the group treated with L-arginine alone serum glucose level was significantly increased from about 6 mM on day 0 to about 8.5 by day 6. Administration of CCompound1 reduced L-arginine-stimulated blood glucose to control level.

It is generally assumed that in this model oxygen- and NO-derived free radicals mediate the effects of L-arginine on inflammation. Inflammatory mediators then induce β-cell death. Accordingly, CCompound1 is able to protect β-cells against the detrimental actions of inflammatory mediators (IL-113, IL-6, and TNF-α, and perhaps others).

TABLE 11
CCcompound1 prevents the stimulating effect of L-arginine on blood
glucose level.

| | Blood Glucose (mM) n = 6 | | |
|---|---|---|---|
| | | | L-arginine + |
| Day | None | L-arginine | CCcompound1 |
| 0 | 6.30 ± 0.29 | 6.06 ± 0.21 | 6.28 ± 0.31 |
| 2 | 6.06 ± 0.21 | 7.26 ± 0.27 | 6.46 ± 0.33 |
| 4 | 6.08 ± 0.35 | 7.62 ± 0.47 | 6.26 ± 0.21 |
| 6 | 5.68 ± 0.38 | 8.54 ± 0.30 | 6.00 ± 0.49 |

Insulin immunostaining: On day 7, the following scores were obtained for insulin staining:

Control: 1.25±0.15

L-arginine alone: 1.75±0.35

L-arginine+CCI 2.45±0.30*

Accordingly, CCompound1 enhanced islet insulin content (P values <0.05) in agreement with protection of islet cell function against the effects of L-arginine. Presently, it is not clear how L-arginine alone was able to slightly increase insulin content; however, it probably represents a compensatory mechanism similar to that can be observed at early phases of development of type 2 diabetes.

The results of the histological determinations are summarized in TABLE 12. The development of acute necrotizing pancreatitis was confirmed by histology. In hematoxylin-eosin stained sections edema, hyperemia, inflammation, acinar vacuolization, necrosis, and tubular transformation were visible in each group. Vacuolization was more pronounced 24 hours, whereas necrosis was observed one week after pancreatitis induction. The animals treated with CCompound1 showed a significant reduction in tissue necrosis. In this group, vacuolization was also stronger than in the group treated with L-arginine alone, leading to the conclusion that CCompound1 slowed down or even stopped the inflammatory process at an earlier time. Tubular transformation and total damage each was also reduced by CCompound1 in the L-arginine induced pancreatitis model. Overall, the data indicate that CCompound1 has protective effects on the pancreas in the L-arginine-induced pancreatitis model, including control of necrotic events. Accordingly, CC compounds may be used to treat such pancreatic diseases as well.

TABLE 12
Summary of effects of CCcompound1 (CC1) on the pancreas
in the L-arginine-induced pancreatitis model.

| | Treatment | | |
|---|---|---|---|
| | | | L-arginine + |
| Histological observations | None | L-arginine | CC1 |
| Hyperemia | 0.50 ± 0.22 | 1.33 ± 0.21 | 1.17 ± 0.17 |
| Edema | 0.17 ± 0.17 | 1.00 ± 0.00 | 1.33 ± 0.21 |
| Inflammation | 0.33 ± 0.21 | 2.00 ± 0.00 | 2.00 ± 0.00 |
| Vacuolization | 0.50 ± 0.22 | 0.17 ± 0.17 | 1.17 ± 0.17 |
| Necrosis | 0.00 ± 0.00 | 2.67 ± 0.21 | 1.25 ± 0.36 |
| Tubular transformation | 0.00 ± 0.00 | 3.00 ± 0.00 | 2.00 ± 0.37 |
| Total damage | 1.00 ± 0.36 | 10.00 ± 0.36 | 7.75 ± 0.70 |

### Example 9

### Effects of CCompound1 and CCompound3 on the Proliferation of AR42J Pancreatic Tumor Cells

The AR42J rat pancreatic tumor cell line, purchased from ATTC (catalog number: CRL-1492), was propagated in modified Ham's medium containing 20% fetal bovine serum. These cells, derived from a transplantable tumor of the exocrine pancreas, contain significant amounts of amylase and other exocrine enzymes and they produce tumors in athymic mice.

For the experiment, cells were split in 96-well plates at 15,000 cells per well. After 24 hours, the medium was changed for fresh medium (0 hour) and various concentrations of CCompound1 and CCompound3 were added and incubations continued for 96 hours. The MTT assay was used to determine the relative number of viable cells. From the data shown in TABLE 13, in the 2-25 μM concentration range CC compounds had only inhibitory effects on the proliferation of pancreatic cancer cells.

TABLE 13
CCcompound1 and CCcompound3 inhibit proliferation of pancreatic
cancer cells.

A₅₄₀

| Concentration (μM) | CCcompound1 | CCcompound3 |
|---|---|---|
| 0, 0 hour | 0.325 ± 0.071 | 0.325 ± 0.071 |
| 0, 96 h | 1.177 ± 0.064 | 1.177 ± 0.064 |
| 2, 96 h | 1.092 ± 0.083 | 1,167 ± 0.078 |
| 5, 96 h | 0.954 ± 0.080 | 0.868 ± 0.093 |
| 10, 96 h | 0.882 ± 0.062 | 0.745 ± 0.052 |
| 25, 96 h | 0.781 ± 0.090 | 0.675 ± 0.064 |

### Relationship Between the In Vivo and In Vitro Effects of CC Compounds,

Based on the experiments with other small compounds, a close estimate can be made to relate the relevance of in vitro effects obtained in the 2-50 μM concentration range to the observed in vivo effects of CC compounds. At the 4.5 mg/kg effective dose that was used in most in vivo experiments, a mouse (weighing about 25 g) is administered ˜112 μg CCompound1. Since a mouse has about 5 ml blood, this would corresponds to about 22 μg/ml or 44 μM concentration if the entire amount of the injected CC compound would be present in the blood. However, (i) not all injected molecules enter the blood stream at the same time, (ii) there is rapid redistribution of small molecules into the tissues, and (iii) there is relatively rapid clearance of small molecules from the blood. Because of all these factors, the peak concentration of injected small compounds rarely exceeds 20% of the absolute maximum (presently 44 μM) value. For example, in the case of dietary resveratrol administered at a dose of 90 mg/kg, a peak concentration in the blood was reached after about 30 min of administration and represented about 15% of the absolute maximum (i.e. if all administered resveratrol molecules would be present in the blood after 30 min of administration) [Ziegler, C. C., Rainwater, L., Whelan, J. and McEntee, M. F. (2003) Dietary resveratrol does not affect intestinal tumorigenesis in Apc^{Min/+} mice. J. Nutr. 134, 5-10]. Thus, most realistically the maximum peak concentration of CC compounds in the blood will not exceed 10 μM and probably will remain below this value. Thus, the in vitro data obtained with 2-10 μM concentrations of CC compounds are expected to be relevant to the in vivo protective effects. As indicated by the presented data, this is the in vitro concentration range in which CC compounds prevented the inhibitory effects of STZ and fatty acids on cell viability and that enhanced survival and proliferation of stem cells.

1. A method of enhancing in humans and other mammals survival and regeneration of endogenous or transplanted endocrine and exocrine pancreatic cells comprising:
administering a CC compound to a subject needing protection of pancreatic cells, the CC compound containing a heterocyclic moiety to which a quaternary ammonium-containing moiety is attached, the CC compound having the following formula: 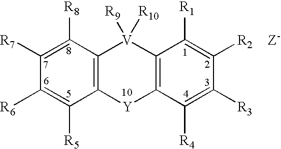
wherein R1 and R3-8 are independently hydrogen, C1-C26 straight, branched or cyclic alkanes or alkenes, aromatic hydrocarbons, alcohols, ethers, aldehydes, ketones, carboxylic acids, amines, amides, nitriles, or five- and/or six-membered heterocyclic moieties;
wherein R9 and R10 considered together are ═O or ═CH-L-N⁺(R11, R12, R13) or
wherein R9 and R10 considered independently are —OH or -L-N⁺(R11, R12, R13);
wherein R2 is represented by the formula: —X or —X′-L-N⁺(R11, R12, R13)Z⁻ or -L-N⁺(R11, R12, R13)Z⁻;
wherein V is —S—, —Se—, —C—, —O— or —N;
wherein Y is —S—, —Se—, —C—, —O— or —N;
wherein X is CH3 or Hydrogen;
wherein —X′ is —CH2-, —OCH2-, —CH2O—, —SCH2- or —CH2S—;
wherein L is a C1-C4 straight alkane, alkene, thiol, ether, or amine;
wherein R11, R12 and R13 are independently C1-C4 straight alkanes, alkenes, thiols,
amines, ethers or alcohols; and
wherein Z- is Cl⁻, Br⁻ or I⁻.

2. The method of claim 1 wherein R11, R12, and R13 are independently methyl, ethyl, propyl, allyl, ether, sulfhydryl, amino, or hydroxyl groups; L is —(CH₂)₂— or —(CH₂)₃—; and R₁ and R₃₋₈ are hydrogen or methyl.

3. The method of claim 1 wherein L-N⁺(R11, R12, R13) is choline.

4. The method of claim 1 wherein the compound is a thioxanthone.

5. The method of claim 4 wherein R9 and R10 considered together are ═O and R2 is —X-L-N⁺(R11, R12, R13)Z⁻.

6. The method of claim 5 wherein the compound is [3-(3,4-dimethyl-9-oxo-9H-thioxanthen-2-yloxy)-2-hydroxypropyl]trimethylammonium chloride.

7. The method of claim 5 wherein the compound is N,N,-diethyl-N-methyl-2-[9-oxo-9H-thioxanthen-2-yl)methoxy]ethanaminium iodide.

8. The method of claim 1 wherein the compound is a thioxanthene.

9. The method of claim 8 wherein R2 is CH₃ or Hydrogen and R9 and R10 considered together are ═CH-L-N⁺(R11, R12, R13); L is —(CH2)2- or —(CH2)3-; and R1 and R3-8 are hydrogen or methyl.

10. The method of claim 8 wherein the compound is N,N,N-trimethyl-3-(9H-thioxanthen-9-ylidene)-propane-1-aminium iodide.

11. The method of claim 8 wherein the compound is N,N-Diethyl-N-allyl-3-(2-methyl-9H-thioxanthen-9-ylidene)-propane-1-aminium bromide.

12. The method of claim 1 wherein the CC compound is administered to a human or another mammal with type 1 diabetes, type 2 diabetes, pancreatitis, pancreatic cancer, or any other disease condition that requires preservation of viability and function of normal endocrine and exocrine pancreatic cells as well as promotion of healing of tissues other than the pancreas that are damaged as a consequence of diabetic state.

13. The method of claim 12 wherein promotion of healing of the corresponding tissue is associated with reduced retinopathy, nephropathy, peripheral neuropathy, cardiomyopathy, and increased wound healing.

14. The method of claim 12 wherein the CC compound directly enhances viability and function of normal pancreatic cells.

15. The method of claim 12 wherein the CC compound indirectly enhances viability and function of normal pancreatic cells and other normal cells damaged by the diabetic condition via enhancing viability and function of stem cells.

16. The method of claim 12 wherein the CC compound indirectly enhances viability and function of pancreatic cells by reducing the proliferation and viability of tumor cells.

17. The method of claim 12 wherein the subject has type 1 diabetes and is administered a CC compound during and/or after receiving islet cell transplantation.

18. The method of claim 17 wherein the islet cells are transplanted together with adult mesenchymal stem cells and hemopoietic stem cells.

19. The method of claim 12 wherein the subject has either type 1 or type 2 diabetes and is administered a CC compound during and/or after receiving stem cell transplantation to support the viability of islet cells and other cells damaged by the diabetic state.

20. The method of claim 19 wherein combined application of a CC compound and stem cell transplantation improves one or more of the following conditions as a consequence of improved islet cell viability: retinopathy, nephropathy, peripheral neuoropathy, cardiomyopathy, cardiovascular disease, and wound healing.

21. The method of claim 18 wherein during isolation, maintenance, and preparation of cells for the transplantation procedure the respective media contains a CC compound to enhance cell viability.

22. The method of claim 21 wherein the concentration of CC compound in the media for the isolation, maintenance, and transplantation of cells is in the range of 2-15 μM.

23. The method of claim 22 wherein the concentration of CC compound in the media is in the range of 2-10 μM.

24. The method of claim 12 wherein the CC compound is administered orally in the form of a tablet, gel capsule, or liquid, or in any other suitable form.

25. The method of claim 24 wherein the CC compound is administered orally at a dose between 100-mg to 2,000-mg per m² body surface of the mammal.

26. The method of claim 24 wherein the CC compound is administered once, twice, or thrice daily, or three-times a week.

27. The method of claim 1 wherein the CC compound is dissolved in a suitable physiologically compatible liquid carrier and administered by either an injection method selected from intravenous, intraarterial, subcutaneous, intraperitoneal, intradermal, or intramuscular, or via infusion, or by using a subcutaneously inserted osmotic minipump to ensure controlled release.

28. The method of claim 27 wherein the CC compound is administered at a dose between 50-mg to 1,000-mg per m² body surface of the mammal once, twice, or thrice daily, or three-times a week.

29. The method of claim 1 wherein the CC compound is administered together, simultaneously, or sequentially with one or more agents used to treat diabetes, and/or pancreatitis, and/or pancreatic cancer.

30. The method of claim 1 wherein the survival of endocrine and exocrine pancreatic cells are at risk due to an inflammatory condition exemplified by but not limited to pancreatitis and pancreatic cancer.

31. The method of claim 1 wherein survival of endocrine islet β-cells is at risk due to high blood levels of glucose, and/or saturated fatty acids or other β-cell damaging lipids.

32. The method of claim 1, wherein one of V or Y is —N, -L-N⁺(R11, R12, R13) and is linked to the —N.

33. The method of claim 1, wherein both of V or Y are —N, -L-N⁺(R11, R12, R13) and is linked to both V and Y.
